(19) United States
(12) Patent Application Publication
(10) Pub. No.: US 2010/0157039 A1
(43) Pub. Date: Jun. 24, 2010

(54) ENDOSCOPE SYSTEM WITH SCANNING FUNCTION

(75) Inventor: Shoji SUGAI, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/644,248

(22) Filed: Dec. 22, 2009

(30) Foreign Application Priority Data
Dec. 22, 2008 (JP) 2008-326361

(51) Int. Cl. A61B 1/04

(52) U.S. Cl. 348/68; 348/E07.085

(57) ABSTRACT

An endoscope system has a light source configured to emit first illumination light and second illumination light; an optical fiber configured to transmit the first and second illumination light to the tip portion of a scope; and scanner configured to spirally scan a target area with the illumination light by vibrating the tip portion of said optical fiber. The endoscope system further has an illumination controller that switches between the first illumination light and the second illumination light in accordance to a scanning position so as to mix areas illuminated by first illumination light with areas illuminated by second illumination light; and an image generator configured to detect pixel signals on the basis of light reflected from the target area at a given sampling rate and to form an observation image from the detected pixel signals. Then, the image generator generates a first observation image from pixel signals created from the first illumination light and generating a second observation image from pixel signals created from the second illumination light.

### BACKGROUND OF THE INVENTION

1. Field of the Invention

The present invention relates to an endoscope system that scans a target area, such as tissue, with illumination light. In particular, it relates to controlling illumination light.

2. Description of the Related Art

An endoscope system with scanning functionality is equipped with a scanning fiber, such as a single mode type of fiber, which is provided in an endoscope. As described in U.S. Pat. No. 6,294,775 and U.S. Pat. No. 7,159,782, the tip portion of the scanning fiber is held by an actuator, such as a piezoelectric device, that vibrates the tip portion spirally by modulating and amplifying the amplitude (waveform) of the vibration. Consequently, illumination light, passing through the scanning fiber, is spirally scanned over an observation area.

Light reflected off the observation area enters into an image fiber and is transmitted to a processor via the image fiber. The transmitted light is transformed to pixel signals by photosensors. Then, each one of the pixel signals detected in time-sequence is associated with a scanning position. Thus, a pixel signal from each pixel is identified and image signals are generated. The spiral scanning is periodically carried out on the basis of a predetermined time interval (frame rate), and one frame's worth of pixel signals are successively read from the photosensors in accordance to a sampling rate.

The number of sampled pixel signals is constant for each spiral scanning revolution. Therefore, in the central portion of an observation image, a length of one revolution is short so that a pixel interval between neighboring detected pixel signals is relatively short compared to the exterior portion of the observation area. Also, pixel information between neighboring pixel signals in the central portion are nearly the same. On the other hand, an interval between image pixels that are two-dimensionally arrayed and constitute an observation image is constant over the entire observation image. Therefore, all of the pixel signals are not raster-arrayed. A portion of the detected pixel signals are utilized to form an observation image, while the remaining pixel signals are abandoned.

### SUMMARY OF THE INVENTION

An object of the present invention is to provide an endoscope system that is capable of obtaining an observation image useful for diagnosis by effectively utilizing detected pixel signals.

An endoscope system according to the present invention has a light source configured to emit first illumination light and second illumination light; an optical fiber configured to transmit the first and second illumination light to the tip portion of a scope; and a scanner configured to spirally scan a target area with the illumination light by vibrating the tip portion of said optical fiber.

The endoscope system further has an illumination controller that switches between the first illumination light and the second illumination light in accordance to a scanning position so as to mix areas illuminated by first illumination light with areas illuminated by second illumination light; and an image generator configured to detect pixel signals on the basis of light reflected from the target area at a given sampling rate and to form an observation image from the detected pixel signals. Then, the image generator generates a first observation image from pixel signals created from the first illumination light and generating a second observation image from pixel signals created from the second illumination light.

In the present invention, areas of the first illumination light and areas of the second illumination light are mixed together in one frame interval, i.e., an interval for scanning an entire area of an observation image. Since a scanning position that are extremely adjacent to one another are illuminated by the first illumination light and the second illumination light, a pixel signal according to the first illumination light and a pixel signal according to the second illumination light can be detected on the substantially same position. Consequently, the first and second observation images, which have the same target area and the substantially same resolution, are created separately.

To scatter the first illumination light and the second illumination light certainly, the illumination controller may alternately switch between the first illumination light and the second illumination light so as to emit a pulse light.

When displaying two images simultaneously, the endoscope system may be equipped with a displaying processor that displays the first observation image and the second observation image simultaneously. Furthermore, the light source may emit third illumination light. The illumination controller may switch between the first illumination light, the second illumination light, and the third illumination light so as to mix together areas illuminated by first illumination light, areas illuminated by second illumination light, and areas illuminated by third illumination light. Also, the image generator may generate a third observation image from pixel signals created from the third illumination light. The displaying processor may display the first observation image, the second observation image, and the third observation image simultaneously.

The light source may any illumination light having specific wavelengths, for example, normal or standard light that forms a full color image, excitation light that forms a fluorescence image, and long-wavelength light included in or adjacent to the infrared spectrum. When diagnosing cancer, white light and excitation light (or nearly infrared light) may be applied.

The sampling rate may be set to a constant rate in each spiral scanning line. However, when detecting excessive pixel signals compared with a resolution necessary for forming an observation image, most of detected pixel signals are abandoned. On the other hand, in the exterior area of the observation image, a pixel interval between detected pixel signals is close to a pixel interval between neighboring image-pixels for forming an observation image.

Therefore, the illumination controller may switch between the first illumination light and the second illumination light in a partial area. In the partial area, the number of detected pixel signals is greater than the number of image-pixels necessary for forming an observation image. In other words, a pixel interval of sampled pixel signals is shorter than a pixel interval of image-pixels. For example, the illumination controller may switch between the first illumination light and the second illumination light in a central part of an entire scanning area. Also, the illumination controller may continuously illuminate the area outside of the partial area with one of the first illumination light and the second illumination light.

Considering that two images may be displayed simultaneously, the partial area may be defined such that a resolution of the first observation image is the same as that of the second observation image. On the other hand, the partial area is defined in accordance to a ratio of the number of abandoned pixel signals to the number of detected pixel signals in one revolution. For example, the partial area is defined such that the ratio is 50% or more than 50%.

To acquire and display various images optionally, the illumination controller may switch between the first illumination light and the second illumination light, as light that illuminates both the partial area and the area outside of the partial area.

When illumination light is like infrared light or nearly infrared light, a distance from a target area can be measured. Therefore, the endoscope may be equipped with a distance-measuring processor that measures a distance from the scope tip portion to the target area. The distance-measuring processor measures the distance with the third illumination light that is long-wavelengths light included in or adjacent to the infrared spectrum.

An apparatus for controlling illumination light, according to another aspect of the present invention, has a light source configured to emit first illumination light and second illumination light; and an illumination controller that controls an emission of the first and second illumination light when spirally scanning a target area with the illumination light by vibrating the tip portion of said optical fiber. The illumination controller switches between the first illumination light and the second illumination light in accordance to a scanning position so as to mix together areas illuminated by the first illumination light with areas illuminated by the second illumination light. Also, an apparatus for forming an observation image, according to another aspect of the present invention, has an pixel signal detector configured to detect pixel signals on the basis of light that is emitted by the above apparatus and that is reflected from the target area at a given sampling rate; and an image generating processor configured to form an observation image from the detected pixel signals. The image generating processor generates a first observation image from pixel signals created from the first illumination light and generating a second observation image from pixel signals created from the second illumination light.

A method for controlling an emission of illumination light, according to another aspect of the present invention, includes: a.) emitting first illumination light and second illumination light; b.) controlling an emission of the first and second illumination light when spirally scanning a target area with the illumination light by vibrating the tip portion of said optical fiber; and c.) switching between the first illumination light and the second illumination light in accordance to a scanning position so as to mix together areas illuminated by the first illumination light with areas illuminated by the second illumination light. A method for forming an observation image, another aspect of the present invention, includes: e.) detecting pixel signals on the basis of light that is emitted by the above method and that is reflected from the target area at a given sampling rate; and f.) generating a first observation image from pixel signals created from the first illumination light and generating a second observation image from pixel signals created from the second illumination light.

### BRIEF DESCRIPTION OF THE DRAWINGS

The present invention will be better understood from the description of the preferred embodiments set forth below, together with the accompanying drawings in which:

FIG. 1 is a block diagram of an endoscope system according to a first embodiment;

FIG. 2 is an illustration of the scanning optical fiber, scanning unit, and spiral scanning pattern;

FIG. 3 illustrates areas of illumination;

FIG. 4 is a timing chart of illumination light;

FIG. 5 is a flowchart of the illumination control process; and

FIGS. 6A and 6B are views of screens in the two-image mode and the three-image mode, respectively.

### DESCRIPTION OF THE PREFERRED EMBODIMENTS

Hereinafter, the preferred embodiments of the present invention are described with reference to the attached drawings.

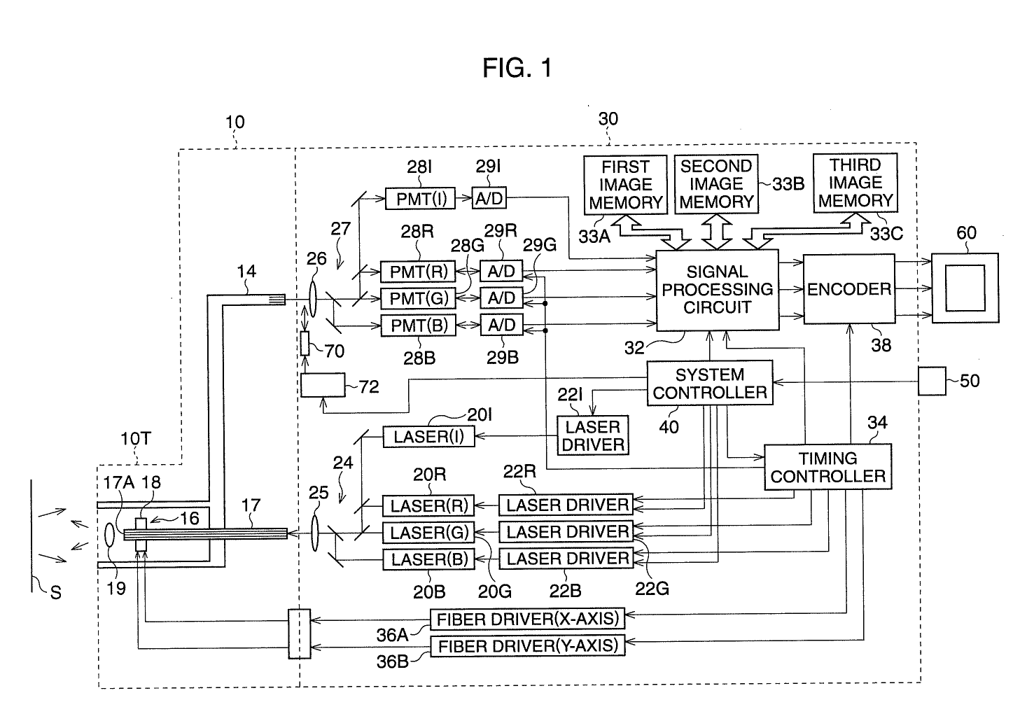

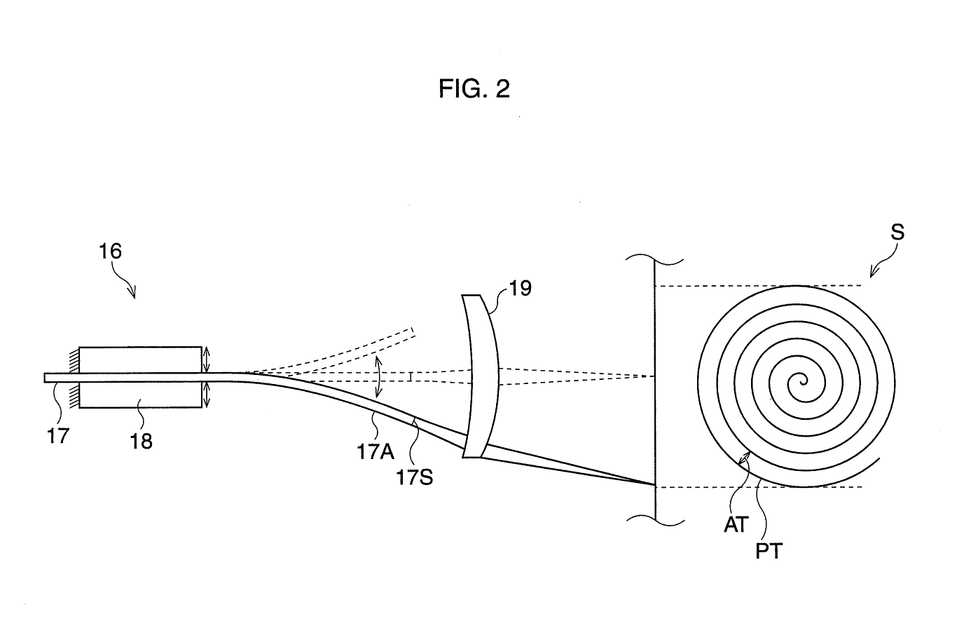

FIG. 1 is a block diagram of an endoscope system according to a first embodiment. FIG. 2 is an illustration of the scanning optical fiber, scanning unit, and spiral scanning pattern.

The endoscope system is equipped with a processor 30 and an endoscope 10 that includes a scanning fiber 17 and an image fiber 14. The single mode type of scanning fiber 17 transmits illuminating light, whereas the image fiber 14 transmits light that is reflected off an observation target S, such as tissue. The image fiber 14 forks around an optical lens 19. The endoscope 10 is detachably connected to the processor 30, and the monitor 60 is connected to the processor 30.

The processor 30 has lasers 20R, 20G, and 20B that emit red, green and blue light, respectively. The lasers 20R, 20G and 20B are driven by laser drivers 22R, 22G and 22B, respectively. The simultaneously emitted red, green, and blue light is collected by half-mirror sets 24 and a collection lens 25. Consequently, white light enters the scanning fiber 17 and travels to the tip portion 10T of the endoscope 10. The light exiting from the scanning fiber 17 illuminates the target S.

Also, the laser 20B can only emit short-wavelength blue light corresponding to “excitation light”. Furthermore, a laser 201, which is driven by a laser driver 221, emits nearly infrared light having long wavelengths close to wavelengths of the infrared spectrum.

As shown in FIG. 2, a scanning unit 16 is provided in the scope tip portion 10T. The scanning unit 16, which has a cylindrical actuator 18, scans the target S with illumination light. The optical fiber 17 passes through the axis of the actuator 18. The fiber tip portion 17A, which cantilevers from the actuator 18, is supported by the actuator 18.

The actuator 18 positioned at the scope tip portion 10T is, herein, a piezoelectric tubular actuator that resonates the fiber tip portion 17A in two dimensions. Concretely speaking, a pair of piezoelectric devices in the actuator 18 vibrates the fiber tip portion 17A with respect to two axes (X-axis and Y-axis) that are perpendicular to one another, in accordance to a resonant mode. The vibration of the fiber tip portion 17A spirally displaces the position of the fiber end surface 17S from the axial direction of the optical fiber 17.

The light emitted from the end surface 17S of the scanning fiber 17 passes through an objective lens 19, and reaches the target S. A course traced by a scanning beam, i.e., a scan line PT, forms a spiral pattern (see FIG. 2). Since a spiral interval AT between adjacent scan lines is tight in a radial direction, the total observation area S is illuminated by spirally scanned light.

Light reflected from the target S enters the image fiber 14 and is transmitted to the processor 30. When the reflected light exits from the image fiber 14, it is divided into R, G, and B light by an optical lens 26 and half-mirror sets 27. The separated R, G, and B light then continues on to photosensors 28R, 28G and 28B, respectively, which transform the R, G, and B light into pixel signals corresponding to colors “R”, “G” and “B”. The pixel signals are detected in accordance to a given sampling rate.

The generated analog pixel signals are converted to digital pixel signals by A/D converters 29R, 29G and 29B before being stored in a first image memory 33A or a second image memory 33B. The stored pixel signals are then fed to a signal processing circuit 32, in which a mapping process is carried out. The successively generated digital R, G, and B pixel signals are arrayed in accordance to the order of a spiral scanning pattern. In the mapping process, each one of the digital R, G, and B pixel signals is associated with a corresponding scanning position, so that raster-arrayed image-pixel signals are formed. Consequently, the pixel position for each of the R, G, and B digital image-pixel signals is identified, in order, and one frame's worth of digital R, G, and B image-pixel signals are generated successively.

In the signal processing circuit 32, the generated two-dimensional image-pixel signals are subjected to various image-processing procedures, including a white balance process to create video signals. The video signals are sent to the monitor 60 via an encoder 37, so that an observation image is displayed on the monitor 60.

In the endoscope system, a plurality of display modes can be set by operating a mode switch 50, which is provided on a front panel of the video processor 30. Herein, three different modes can be selected: a normal observation mode for obtaining a full-color image (normal/standard image); a two-image mode for obtaining a full-color image and a fluorescence image; and a three-image mode for obtaining a full-color image, a fluorescence image, and a (nearly) infrared image.

When the two-image mode is selected, the target S is illuminated with alternating white light and excitation light. Thus, reflected white light and fluorescence both enter the scope tip portion 10T on an alternating basis. A filter 70 provided in the scope tip portion 10T is selectively positioned with respect to the path of the light exiting the image fiber 14. During the interval of illumination by excitation light, the elimination filter 70 is re-positioned from outside of the optical path to directly within the optical path by an actuator 72. Thus, reflected excitation light is eliminated while reflected alternating white light and fluorescence reach the photo-sensors 28R, 28G, and 28B. Pixel signals based on the white light and pixel signals based on the fluorescence are generated on an alternating basis and temporarily stored in the first image memory 33A and the second image memory 33B, respectively. Then, video signals based on the white light and video signals based on the excitation light are output to the monitor 60, so that a normal observation image and a fluorescence image are displayed simultaneously.

When the three-image mode is selected, white light, excitation light and infrared light are emitted on an alternating basis. A photo-sensor 281 transforms the reflected light to pixel signals, and the detected pixel signals are temporarily stored in a third image memory 33C. In the signal processing circuit 32, image-pixel signals based on the infrared light are generated in addition to the image-pixel signals based on white light and the image-pixel signals based on fluorescence. Thus, a normal image, a fluorescence image and an infrared image are all displayed on the monitor 60, simultaneously.

A system controller 40, which includes a ROM unit, a RAM unit, and a CPU, controls the action of the video processor 30 and the videoscope 10 by outputting control signals to the signal processing circuit 32, a timing controller 34, and the laser drivers 22R, 22G, 22B, and 221, etc. A control program is stored in the ROM unit. The timing controller 34 outputs synchronizing signals to fiber drivers 36A and 36B for driving the scanning unit 16, and to the laser drivers 22R, 22G, 22B, and 221 to synchronize the vibration of the tip portion 17A with the timing of the emission of light.

The output of lasers 20R, 20G, 20B, and 201 is controlled by driving signals fed from the laser drivers 22R, 22G, 22B, and 221. Thus, an amount of illumination light (intensity of light) that is incident on a target is adjustable. In the signal processing circuit 32, luminance signals are generated from the digital image-pixel signals and then transmitted to the system controller 40. The system controller 40 outputs control signals to the laser drivers 22R, 22G, 22B, and 221 to adjust the amount of illumination light. Thus, a proper brightness is maintained.

In the case of the three-image mode, the system controller 40 measures a distance from the scope tip portion 10T to the target S on the basis of image-pixel signals obtained from infrared light. Then, the system controller 40 uses the detected distance to adjust the intensity of excitation light to control the amplification of pixel signals created from fluorescence. As a result, by referring to the distance an operator can diagnose whether or not a dark portion of a fluorescent image is tissue.

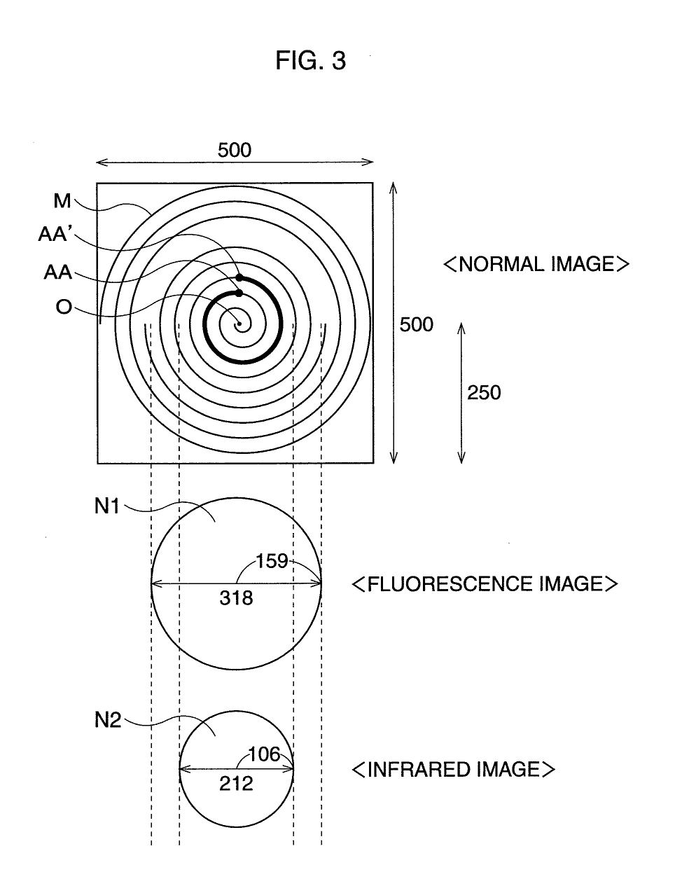

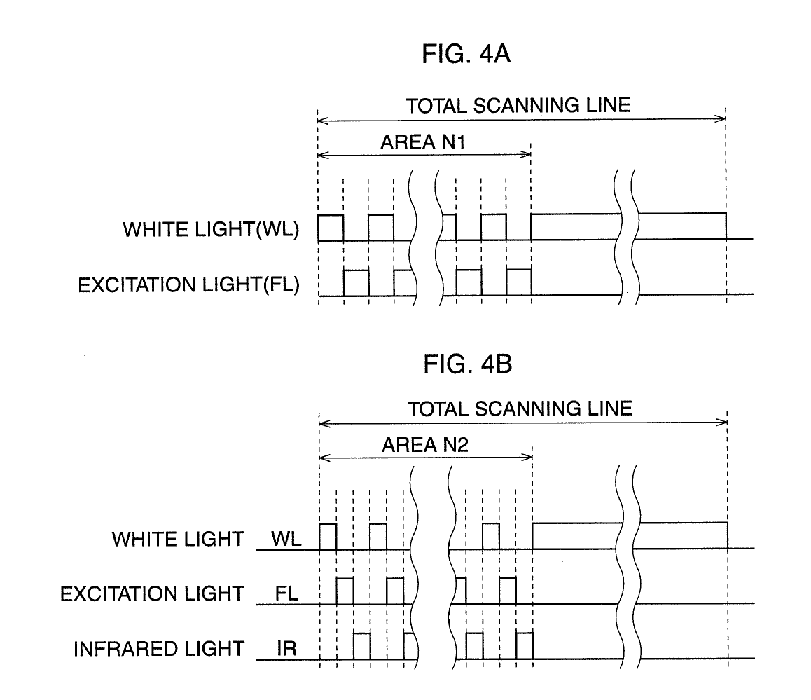

FIG. 3 illustrates areas of illumination. FIG. 4 is a timing chart of illumination light.

One frame's worth of a circular observation image is formed by a spiral scan, and the number of scan lines in a radial direction depends on the number of spiral revolutions. Note that a scanning section from one scan point on a given straight line to another scan point on the same straight line extending radially outward, where the two points are separated by one 360-degree spiral scanning revolution, is herein counted as “one scan line” (see scan line AA-AA′ in FIG. 3).

In the normal observation mode, an observation image corresponding to an entire scanning area M is displayed with the resolution of “500×500” image pixels (dots). In other words, 250 pixels are arrayed from a center point “0”, which corresponds to a scan starting point, to a point on the exterior of the scanning pattern in the radial direction.

Pixel signals are generated by the photo-sensors 28R, 28G, and 28B at a predetermined sampling rate. Herein, the number of sampled pixels in each revolution (one spiral) is constant. For example, the number of samples is set to 2000/spiral. The angular velocity of a spiral scan is also constant. Therefore, a pixel interval between neighboring pixel signals in the central part of the scanning area M is so short that neighboring pixel signals are superposed on one another. This is because the length of one revolution is relatively short. On the other hand, an interval between neighboring pixel signals in the exterior portion is similar to an interval between the image pixels that constitute the observation image. Namely, the interval is appropriate for realizing the resolution of “500×500” dots. Therefore, in the normal observation mode only a portion of the pixel signals detected in the central area are selected or sampled to constitute the observation image.

On the other hand, in the two-image mode the central area is illuminated with both white light and excitation light on an alternating basis to form a pulse light. However, the area outside of the central area is illuminated with only white light. The size of the central area, which is smaller than the entire scan area M, is defined such that a resolution of a normal image is the same as that of a fluorescence image. The size of the central area that is illuminated by both white light and excitation light is determined as follows.

When the sampled pixel signals number 2000, a scanning line that can form image pixels by using only-half of the 2000 (=1000) pixel signals is obtained from the following formula. Note that a length of one revolution is designated by “I”, which also corresponds to the number of pixel signals used when the pixel signals are tightly arrayed along a scanning line. A radius of the scan line to be obtained is herein designated by “r”.

I=2000/2=2×π×r (1)

r=159 is calculated from the formula (I).

When an interval between scanning lines along a radial direction is tight, the radius “r” of a particular scanning line substantially corresponds to the number of spirals inside of that particular line. Therefore, in an area N1 having a radius r=159, namely, an area N1 that includes the “159” spiral lines, an observation image can be formed by one-half or less of the detected pixel signals. In other words, more than half of the detected pixel signals are substantially overlapping one another. Hence, when emitting alternating white light and excitation light within the area N1, an image obtained from the white light and an image obtained from the excitation light, both of which have the same resolution, can be generated.

In FIG. 4A, the timing of illumination in the two-image mode is illustrated. After scanning starts, the area N1 is illuminated by alternating white light (WL) and excitation light (FL). But once the scanning point passes outside of the area N1, the exterior area is illuminated by white light only.

On the other hand, in the case of the three-image mode, an area smaller than area N1 is illuminated by alternating white light, excitation light, and nearly infrared light (IR). When the sampling rate is 2000/spiral, a scanning line that can form image pixels by using only one-third of the 2000 pixel signals is obtained from the following formula.

I=2000/3=2×π×r (2)

The radius r=106 is obtained from the above formula. Therefore, an area N2 that encompasses the “102” innermost spirals is illuminated by alternating white light, excitation light and nearly infrared light. In FIG. 4B, illumination timing for the three-image mode is illustrated.

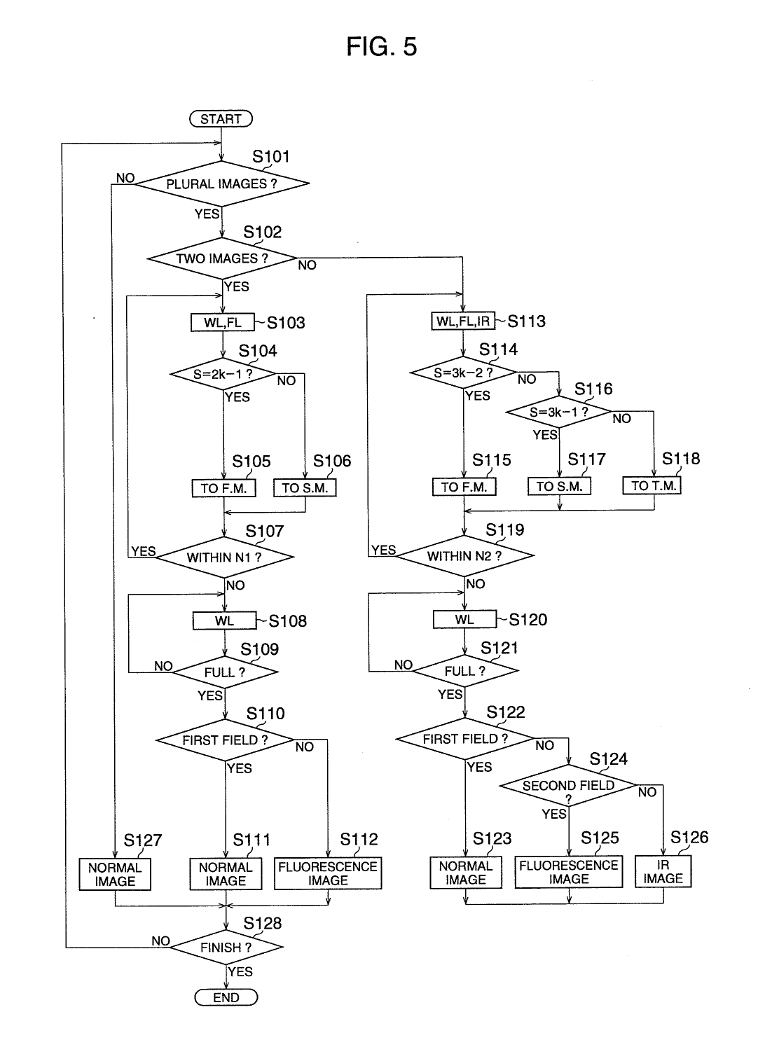

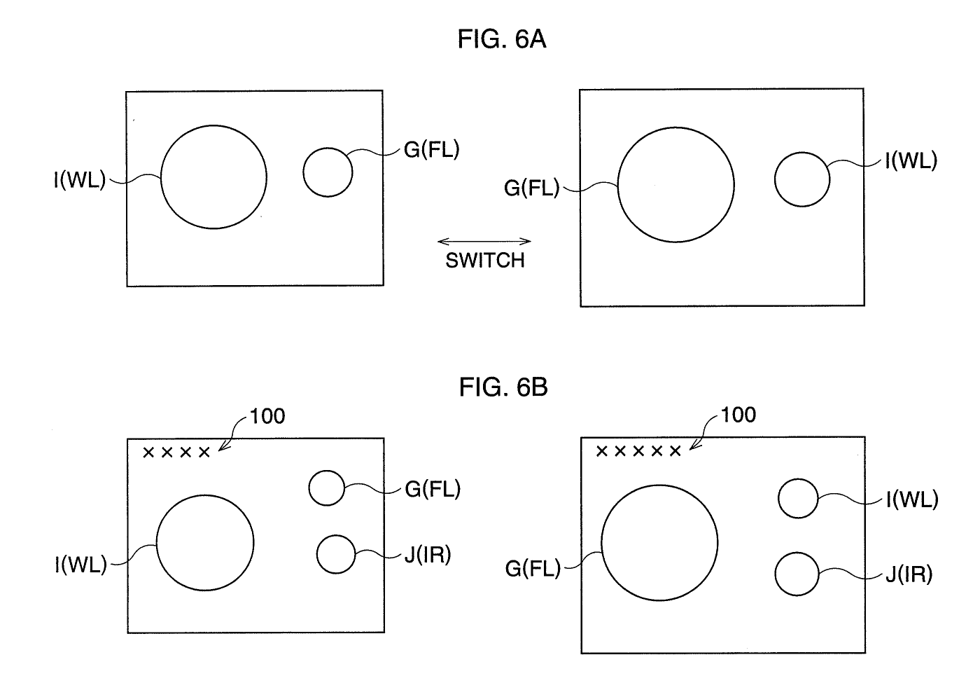

FIG. 5 is a flowchart of the illumination control process. FIGS. 6A and 6B are views of screens in the two-image mode and the three-image mode, respectively.

In Step S101, it is determined whether the two-image mode or three-image mode has been selected by an operator. When the normal observation mode is set, the entire scan area is illuminated by white light (WL) only (Step S127), and a standard, full-color image is displayed on the entire screen of the monitor 60. On the other hand, when the two-image mode or three-image mode is selected, the process proceeds to Step S102.

In Step S102, it is determined whether the two-image mode has been selected. When the two-image mode is selected, the timing controller 34 controls the laser drivers 22R, 22G, and 22B so as to emit white light (WL) and excitation light (FL) on an alternating basis (Step S103). The laser drivers 22R, 22G, and 22B switch between simultaneous emission of R, G, and B light and the emission of short-wavelength light in accordance to the sampling rate (=2000/spiral).

In Step S104, the number of samples is counted on the basis of the sampling rate. The number of samples “SS” corresponds to a sampled pixel position. When the sampled pixel position is an odd number (=2k−1), the pixel position is illuminated by white light. On the other hand, when the sampled pixel position is an even number (=2k), the pixel position is illuminated by excitation light. Pixel signals detected from odd-number positions are stored in the first image memory 33A (Step S105), whereas pixel signals detected from even-number positions are stored in the second image memory 33B (Step S106).

In Step S107, it is determined whether a present scanning position is within the area N1 shown in FIG. 3. While scanning the inside of the area N1, Steps S103 to S106 are repeated. On the other hand, when a present scanning position is outside of the area N1, the process goes on to Step S108.

In Step S108, the laser drivers 22R, 22G, and 22B are controlled so as to emit white light continuously; detected pixel signals are stored in the first image memory 33A, and the process of Step S108 continues until the entire scan area is illuminated (S109).

Note that, in the area N1, there are an excess number of pixel signals that are not necessary for forming a normal image and a fluorescence image. This is because the number of spirals in the area N1 is less than the number “159” spirals containing the full amount of pixel signals that are substantially used to form both images. These extra pixel signals are abandoned. Redundant pixel signals outside of the area N1 are also not used.

In the signal-processing circuit 32, image-pixel signals of the standard image and image-pixel signals of the fluorescence image are generated and then stored temporarily in the first image memory 33A and the second image memory 33B, respectively. Image-pixel signals for the normal image are output to the signal-processing circuit 32 in a first field interval, whereas image-pixel data for the fluorescence image are output to the signal-processing circuit 32 in a second field interval (Step S110 to S112).

In FIG. 6A, the screen displaying the two-image mode is shown. A normal image I (WL) based on white light is the size of the entire scan area M. A fluorescence image G (FL) based on excitation light has a size corresponding to the scan area N1 that is smaller than the complete scan area M.

On the other hand, when it is determined at Step S102 that the three-image mode is selected, the process progresses to Step S113. In Step S113, the laser drivers 22R, 22G, 22B, and 221 are controlled so as to emit white light, excitation light, and nearly infrared light on an alternating basis. Switching between emission sources is carried out in synchronicity with the timing of the detected pixel signals based on the sampling rate.

Detected pixel signals are divided into three groups; i.e., pixel signals based on white light, pixel signals based on fluorescence, and pixel signals based on nearly infrared light, in accordance to the sample number SS. These three groups of pixel signals are stored in the first memory 33A, the second memory 33B, and the third memory 33C, respectively (Steps S114 to S118).

While the area N2 is being scanned, Steps S113 to S118 are repeated (Step S119). When the scanning position moves outside of the area N2, the lasers 20R, 20G, and 20B are controlled to emit only white light and detected pixel signals are stored in the first memory 33A (Step S120). Note that redundant pixel signals are abandoned similarly to the two-image mode.

Step S120 continues until scanning of the entire scan area is finished (Step S121). Three groups of image-pixel signals are output at three field intervals. Image-pixel signals of a normal image are output at a first field interval, image-pixel signals of a fluorescence image are output at a second field interval, and image-pixel signals of an infrared image are output at a third field interval (Step S122 to S126). Steps S101 to S127 are repeated until an observation is finished (Step S128).

In FIG. 6B, the screen in which a normal image I (WL), a fluorescence image G (FL), and an infrared image J (IR) are displayed simultaneously is shown. The size of the fluorescence image G and the infrared image J corresponds to the size of the scan area N2 shown in FIG. 3. In the three-image mode, in addition to the display of three images, the distance from the fiber tip portion to the target is measured and di stance information 100 is also displayed on the screen.

Furthermore, when an illumination switch (not shown), which is provided on the processor 30, is operated during the two-image mode or three-image mode, illumination light for both the central area and the area outside of the central area is changed. In the case of the two-image mode, excitation light instead of white light is emitted at Step S108. As a result, a fluorescence image corresponding to the size of the entire scan area M and a normal image corresponding to the area N1 are displayed (See FIG. 6A). In the case of the three-image mode, excitation light instead of white light is emitted at Step S120. Thus, a fluorescence image having the size of the entire scan area M is displayed (see FIG. 6B).

In this way, in the present embodiment, illuminating light is spirally scanned by vibrating the fiber tip portion two-dimensionally. Then, in the two-image mode, alternating white light and excitation light are emitted in the area N1, and white light is emitted outside of the area N1. In the three-image mode, white light, excitation light, and nearly infrared light are emitted on an alternating basis in the area N2, and white light is emitted outside of the area N2

In either area N1 or N2 where many pixel signals overlap one another, two images or three images that are of a different type from one another are displayed simultaneously with the same resolution. Namely, a plurality of images that are useful for a diagnosis can be displayed simultaneously. Furthermore, an operator can diagnose tissue by referring to the distance from the scope tip portion to the tissue.

A combination or blend of different types of illuminating light may be selected in the two-image or three-image modes. For example, excitation light and nearly infrared light may be emitted in the two-image mode. Furthermore, illuminating light other than the above light may be emitted. For example, light having a narrow-band wavelength for observing the blood of a mucous membrane may be emitted.

The size of scanning areas N1 and N2 may be optionally defined in accordance to the resolution of an observation image, the sampling rate, etc. Also, in an area where many pixel signals overlap and are redundant, illuminating light may be emitted so as to mix together areas illuminated by one light with areas illuminated by the other light, instead of emitting illuminating light on an alternating basis. As for the scanning method, illuminating light may be scanned by driving an optical lens.

The present disclosure relates to subject matter contained in Japanese Patent Application No. 2008-326361 (filed on Dec. 22, 2008), which is expressly incorporated herein, by reference, in its entirety.

1. An endoscope system comprising:
a light source configured to emit first illumination light and second illumination light;
an optical fiber configured to transmit the first and second illumination light to the tip portion of a scope;
a scanner configured to spirally scan a target area with the illumination light by vibrating the tip portion of said optical fiber;
an illumination controller that switches between the first illumination light and the second illumination light in accordance to a scanning position so as to mix areas illuminated by first illumination light with areas illuminated by second illumination light; and
an image generator configured to detect pixel signals on the basis of light reflected from the target area at a given sampling rate and to form an observation image from the detected pixel signals, said image generator generating a first observation image from pixel signals created from the first illumination light and generating a second observation image from pixel signals created from the second illumination light.

2. The endoscope system of claim 1, wherein said illumination controller switches between the first illumination light and the second illumination light in a partial area in which a greater number of pixel signals is detected than the number of image-pixels necessary for forming an observation image.

3. The endoscope system of claim 2, wherein the partial area is defined such that a resolution of the first observation image is the same as that of the second observation image.

4. The endoscope system of claim 2, wherein the partial area is defined in accordance to a ratio of the number of abandoned pixel signals to the number of detected pixel signals in one revolution.

5. The endoscope system of claim 2, wherein said illumination controller switches between the first illumination light and the second illumination light in a central part of an entire scanning area.

6. The endoscope system of claim 2, wherein said illumination controller continuously illuminates the area outside of the partial area with one of the first illumination light and the second illumination light.

7. The endoscope system of claim 6, wherein said illumination controller switches between the first illumination light and the second illumination light, as light that illuminates both the partial area and the area outside of the partial area.

8. The endoscope system of claim 1, wherein said illumination controller alternately switches between the first illumination light and the second illumination light so as to emit a pulse light.

9. The endoscope system of claim 1, further comprising a displaying processor that displays the first observation image and the second observation image simultaneously.

10. The endoscope system of claim 1, wherein the first illumination light and the second illumination light are two components of normal light that forms a full color image, excitation light that forms a fluorescence image, and long-wavelength light included in or adjacent to the infrared spectrum.

11. The endoscope system of claim 1, wherein said light source emits third illumination light, said illumination controller switching between the first illumination light, the second illumination light, and the third illumination light so as to mix together areas illuminated by first illumination light, areas illuminated by second illumination light, and areas illuminated by third illumination light, said image generator generating a third observation image from pixel signals created from the third illumination light.

12. The endoscope system of claim 11, wherein said illumination controller alternately switches between the first illumination light, the second illumination light, and the third illumination light in a pulse sequence.

13. The endoscope system of claim 11, further comprising a displaying processor that displays the first observation image, the second observation image, and the third observation image simultaneously.

14. The endoscope system of claim 11, further comprising a distance-measuring processor that measures a distance from the scope tip portion to the target area, said distance-measuring processor measuring the distance with the third illumination light that is long-wavelengths light included in or adjacent to the infrared spectrum.

15. An apparatus for controlling illumination light, comprising:
a light source configured to emit first illumination light and second illumination light; and
an illumination controller that controls an emission of the first and second illumination light when spirally scanning a target area with the illumination light by vibrating the tip portion of said optical fiber, said illumination controller switching between the first illumination light and the second illumination light in accordance to a scanning position so as to mix together areas illuminated by the first illumination light with areas illuminated by the second illumination light.

16. An apparatus for forming an observation image, comprising:
an pixel signal detector configured to detect pixel signals on the basis of light that is emitted by the apparatus recited claim 15 and that is reflected from the target area at a given sampling rate; and
an image generating processor configured to form an observation image from the detected pixel signals, said image generating processor generating a first observation image from pixel signals created from the first illumination light and generating a second observation image from pixel signals created from the second illumination light.

17. A method for controlling an emission of illumination light, comprising:
emitting first illumination light and second illumination light;
controlling an emission of the first and second illumination light when spirally scanning a target area with the illumination light by vibrating the tip portion of said optical fiber; and
switching between the first illumination light and the second illumination light in accordance to a scanning position so as to mix together areas illuminated by the first illumination light with areas illuminated by the second illumination light.

18. A method for forming an observation image, comprising:
detecting pixel signals on the basis of light that is emitted by the method recited in claim 17 and that is reflected from the target area at a given sampling rate; and
generating a first observation image from pixel signals created from the first illumination light and generating a second observation image from pixel signals created from the second illumination light.
